(19) United States
(12) Patent Application Publication
(10) Pub. No.: US 2024/0015486 A1
(43) Pub. Date: Jan. 11, 2024

(54) AUTOMATIC DENTAL DEVICE MANAGEMENT SYSTEM

(71) Applicant: 3M INNOVATIVE PROPERTIES COMPANY, St. Paul, MN (US)

(72) Inventors: Rudolf Schmid, Eichenau (DE); Wolfgang Weinmann, Weilheim (DE)

(21) Appl. No.: 18/025,567

(22) PCT Filed: Aug. 5, 2021
(86) PCT No.: PCT/IB2021/057223

§ 371 Date: Mar. 9, 2023

Related U.S. Application Data
(60) Provisional application No. 63/076,651, filed on Sep. 10, 2020.

(51) Int. Cl. H04W 4/38; G16H 40/40; G16H 40/67; A61C 13/15; A61C 5/60; B33Y 50/02; B33Y 80/00; B33Y 30/00

(52) U.S. Cl. CPC H04W 4/38; G16H 40/40; G16H 40/67; A61C 19/003; A61C 5/60; B33Y 50/02; B33Y 80/00; B33Y 30/00; A61C 2204/002

(57) ABSTRACT

A dental device system includes a dental device configured to transmit data via a narrowband network to a computing device that outputs a notification indicative of a device management operation. The dental device system includes a computing device and a dental device. The dental device includes a sensor configured to generate a signal indicative of a condition of the dental device and communication circuitry configured to receive the signal from the sensor and transmit data indicative of a health of the dental device via a narrowband network to the computing device. The computing device is configured to determine, based on the data indicative of the health of the dental device, the health of the dental device; determine, based on the health, a device management operation; and output a notification indicative of the device management operation.

### TECHNICAL FIELD

The present disclosure relates to the field of dental devices, including dental devices that generate data.

### BACKGROUND

Dental clinicians use a variety of dental devices such as curing light devices, mixing machines, or other equipment configured to process, make, or use materials for procedures in endodontics, orthodontics, periodontics, prosthodontics, and oral surgery. Typically, dental devices operate in a standalone manner. For example, information created by the dental device, such as by sensors or integrated processors, is provided to the device operator, such as by integrated display. As one example, a battery charge indicator may be included on a dental curing light device.

### SUMMARY

The disclosure describes systems, dental devices, and techniques that include transmitting data indicative of a condition of a dental device, such as via a narrowband network, to determine a health of the dental device. The health of the dental device is used to determine a device management operation and output a notification indicative of the device management operation. The device management operation may, for example, be used for localization of the dental device, initialize remote maintenance of the dental device or a component thereof, initialize repair or replacement of the dental device or a component thereof, improve the condition of the dental device or a component thereof, or deliver a recommendation to a user. The user may include an operator of the dental device, such as a dental clinician, a service technician, a service center, or a manufacturer of the dental device system, for example. By determining the device management operation, the systems, dental devices, and techniques may reduce time to repair or replace the dental device, reduce device malfunction, and/or reduce misuse of the dental device by providing timely analytic information to a user. These potential benefits may increase dental clinician confidence and satisfaction with the dental device and servicing of the dental device, reduce the cost of the dental device and servicing of the dental device, and/or enable a manufacturer to improve the dental device to meet dental clinician requirements and expectations.

In one example, this disclosure is directed to a dental device system that may include a computing device and a dental device. The dental device may include a sensor configured to generate a signal indicative of a condition of the dental device and communication circuitry configured to receive the signal from the sensor and transmit data indicative of a health of the dental device via a narrowband network to the computing device. The computing device may include processing circuitry configured to receive the data indicative of the health of the dental device via the narrowband network and a memory including instructions that, when executed by the processing circuitry, cause the processing circuitry to determine, based on the data, the health of the dental device, then determine, based on the health, a device management operation, and output a notification indicative of the device management operation.

In another example, this disclosure is directed to a dental device that includes a sensor configured to generate a signal indicative of a condition of the dental device, processing circuitry configured to receive, from the sensor, the signal, and process the signal to generate data indicative of a health of the dental device, and communication circuitry configured to transmit the data via a narrowband network.

In another example, this disclosure is directed to a computing device that includes processing circuitry and a memory including instructions that, when executed by the processing circuitry, cause the processing circuitry to receive data indicative of a condition of a dental device via a narrowband network, determine, based on the data, a health of the dental device, determine, based on the health, a device management operation, and output a notification of the device management operation.

In another example, this disclosure is directed to a dental device system that includes a curing light device with a light source and a sensor configured to generate a signal indicative of a condition of the curing light device, the curing light configured to cause the light source to emit optical data based on the signal; and a base station configured to receive the curing light device, the base station including a light sensor configured to, when the curing light device is received in the base station, detect the optical data emitted from the light source of the curing light device.

In a further example, this disclosure is directed to a method that includes, transmitting, by a dental device system comprising at least one dental device, data indicative of a condition of the dental device via a narrowband network to processing circuitry, determining, by the processing circuitry, based on the data, a health of the dental device, further determining by the processing circuitry, based on the health, a device management operation, and outputting, by the processing circuitry, a notification indicative of the device management operation.

The systems and techniques of this disclosure provide one or more technical improvements over existing standalone operation of dental devices. As an example, the systems of this disclosure may allow for reduced service and repair downtimes of dental devices and enable a manufacturer to reduce device malfunction and misuse through the output of timely analytic information to a user.

The details of one or more examples of this disclosure are set forth in the accompanying drawings and the description below. Other features, objects, and advantages of the techniques of this disclosure will be apparent from the description, drawings, and from the claims.

### BRIEF DESCRIPTION OF THE DRAWINGS

FIG. 1 is a block diagram illustrating an example dental device system in which a dental device transmits data via a narrowband network to a computing device.

FIG. 2 is a block diagram illustrating in further detail the example dental device system of FIG. 1.

FIG. 3 illustrates an example dental device system including a curing light device that transmits data via a light source to a base station.

FIG. 4 is a flow chart illustrating an example method for outputting a notification indicative of a device management operation for a dental device.

Numerous other modifications and changes to the examples set forth in this disclosure can be devised by those skilled in the art without departing from the scope and spirit of the principles of this disclosure.

### DETAILED DESCRIPTION

Dental clinicians use a variety of dental devices such as curing light devices, mixing machines, or other equipment to process, make, or use materials for procedures. Typically, dental devices operate in a standalone manner. For example, in standalone operation, data gathered by sensors of the dental device may be provided only to a device operator. In some instances, the device operator ignores the information, or does not understand the information. Ignoring or misunderstanding information displayed by the dental device to the device operator can lead to component degradation or failure, and/or loss of performance or failure of the dental device. In one such example, a dental curing light device light intensity diminishes over time, leading to improper curing of dental materials. Ignoring or misunderstanding information displayed by the dental device also may lead to increased service costs or a decrease in customer satisfaction. Additionally, in some instances, repair of dental devices can lead to prolonged periods of device downtime due to long lead times for service components. This may lead to lost productivity for a dental clinician.

In some examples, data generated by sensors within a dental device may be used by manufacturers, retailers, service centers, and service technicians to reduce service and/or repair downtimes of the dental devices by identifying and/or predicting issues with an operation of the dental device, to track analytic and/or historical information such as malfunction and misuse, or both. With standalone operation, manufacturers, retailers, service centers, and service technicians cannot fully understand component life expectancy within a dental device, or proactively repair dental devices to ensure a device operator (e.g., a dental clinician) does not experience dental device malfunction or dissatisfaction with use of the product. Providing access to dental device data may enable the manufacturer to develop more robust dental device components, ensure better quality of dental devices, and increase operator satisfaction.

As described herein, dental devices may be communicatively coupled to processing circuitry via narrowband network (e.g., narrowband communication), which may facilitate gathering and processing dental device data. Narrowband communication uses radio waves that may permit extensive coverage compared to other types of radio communication. For example, narrowband communication may penetrate through concrete walls and floors, allowing for dental devices within buildings to connect to a narrowband network. Narrowband communication may also use less energy than other types of radio communication. For example, a small battery may power narrowband network communication circuitry within a dental device for at least one year, such as at least ten years. Dental devices including narrowband network communication circuitry can allow device manufacturers, retailers, service centers, or service technicians to remotely monitor the operational life of components, ensure service procedures are scheduled and performed appropriately before a dental clinician experiences device degradation and/or failure, and increase brand loyalty and customer satisfaction.

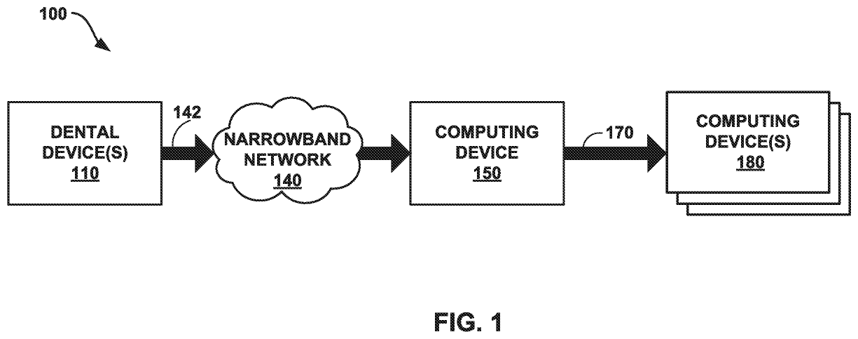

FIG. 1 is a schematic diagram illustrating an example dental device system 100 in which a dental device 110 transmits data 142 via a narrowband network 140 to a computing device 150. The computing device 150 outputs a notification 170 to one or more computing devices 180. The dental device 110 may include one or more dental devices. Example dental device 110 include, but is not limited to, a curing light, three-dimensional printer, mixing machine, post-processing station, or other dental devices, either individually or in combination. Although describe as dental devices used in dental care, in other example, dental device 110 may include other devices, such as, for example, medical devices.

In some examples, dental device 110 may include a curing light dental device. Example curing light dental devices may include Elipar™ curing light or Paradigm™ curing light, both available from 3M Company, St. Paul, Minnesota. A curing light dental device may include, for example, a light source for curing dental materials and a battery to power the curing light dental device.

In some examples, dental device 110 may include a mixing machine dental device. Example mixing machine dental devices may include Pentamix™ automatic mixing unit, available from 3M Company, St. Paul, Minnesota. A mixing machine dental device may include, for example, at least one motor to drive a gear-drive or an output shaft to mix at least two materials. In some examples, dental device 110 may include a post-processing station. The post-processing station may be configured to clean or sanitize dental products prior to or after use, such as implants, prosthetics, restoratives, orthodontic components, crowns, fillings, bridges, braces, aligners, retainers, models, or other dental products, and/or dental instruments, such as mirrors, probes, retractors, handpieces, lasers, wrenches, burs, excavators, burnishers, pluggers, scalers, curettes, or other dental instruments. The post-processing station may be configured to clean dental products and/or dental instruments using either individually, or in combination, a fluid, heat, abrasion, or radiation.

In some examples, dental device 110 may include a three-dimensional printer dental device. The three-dimensional printer dental device may further include one or more printheads and one or more material cartridges configured to manufacture a dental component via additive manufacturing. In some examples, dental devices 110 may include other dental devices or medical devices.

Dental device 110 includes one or more sensors configured to monitor a condition of dental device 110. The condition of dental device 110 may include any suitable metric associated with dental device 110 that is capable of transmission as an analog or digital electronic signal. For example, the condition of dental device 110 may include a condition of dental device 110 or any component of dental device 110, such as, for example, operational status (e.g., on, off, or charging), usage, age, operating hours, temperature, pressure, energy consumption, voltage drop, current draw, impedance, light output, mechanical impact, mechanical noise, material consumption (e.g., via radio frequency identification (RFID) reader), intrusion of fluids, alarms, or error signals.

The condition of dental device 110 may be indicative of a health of dental device 110. For example, the health of dental device 110 may include any suitable metric associated with the performance of dental device 110. In some examples, the health of dental device 110 may be unimpaired. For example, when an operation of dental device 110 is acceptable in view of manufacturing tolerances and/or use tolerances. In some examples, the health of dental device 110 may be impaired. For example, when an operation of dental device 110 is less than acceptable in view of manufacturing tolerances and/or use tolerances.

In examples in which dental device 110 includes a curing light dental device, the condition may include, for example, at least one of a remaining battery life of the battery, a total number of charging cycles of the battery, a battery voltage, a mechanical impact to the curing light device, a temperature around the battery or around the light source or around electronics of the dental device, a luminance of the light source, a number of on/off cycles of the light source, a duration of operation of the curing light device, or other device condition. Analysis of data indicative of such conditions may include, for example, determining a remaining battery life, determining if the curing light has been dropped to the floor, determining if the device has overheated, determining is a light guide is damaged, providing an offer for service of parts such as battery or light guide or protection sleeves, terminating a guarantee or a warranty due to too high mechanical impact, or offering a new curing light after a certain period of use.

In examples in which the dental device 110 includes a mixing machine dental device, the condition may include, for example, at least one of a mechanical impact to the mixing machine, a temperature of the motor or the gear-drive or motor controller circuitry, a duration of operation of the motor or the gear-drive, the acoustics of the device, a pressure at the respective material canisters, a type of material extruded through the mixer, or other device condition. Analysis of data indicative of such conditions may include, for example, determining a remaining life of a motor or a gear drive, determining if the mixing machine has been dropped to the floor, determining if the device has overheated, providing an offer for service of parts such as a gear drive, terminating a guarantee or a warranty due to too high mechanical impact, or offering a new device after a certain period of use, determining how often non-recommended impression materials are used with the device, determining which types of impression materials are preferred by the customer, determining which mix of impression materials are used by the customer, providing offers about material or material bundles, or providing service offers for the device or new device offer with discount.

In examples in which dental device 110 includes a post-processing station, the condition may include, for example, at least one of a mechanical impact to the post-processing station, a temperature of the motor or the gear-drive or motor controller circuitry, a duration of operation of the motor or the gear-drive or light source or heat source, acoustics of the dental device, a type of cleaning fluid used in the post-processing station, a count of cleaning cycles, a duration of cleaning cycles, or a pressure or vacuum within post-processing station, or other device condition. Analysis of data indicative of such conditions may include, for example, calculating an amount of consumable material used, determining use of non-recommended material, calculating a number of cleaned parts, determining LEDs have reach the end of useable life, generating warnings to reduce complaints if the device is not used according specification, providing offers for service of parts such as LED arrays, gas cartridges, or consumable materials, and/or providing service offers for the device or new device offer with discount.

In examples in which dental device 110 includes a three-dimensional printer, the condition may include at least one of a mechanical impact to the three-dimensional printer, a type of material cartridge used in the three-dimensional printer, a leakage of the cartridge or printer, a temperature of the printing head or inside the printer, acoustics associated with movement inside the printer, a luminance of the light source of the three-dimensional printer or a duration of operation of the three-dimensional printer, or other device condition. Analysis of data indicative of such conditions may include, for example, calculating a number of printed parts, logging used program parameters, generating warnings if parameters such as nozzle temperature or curing time do not fit with used printing material, determining LEDs reach the end of useable life, determining failure in optics, generating warnings to reduce complaints if device is not used according specification, providing offers for service of parts such as led array or consumable material, determining use of non-recommended material, determining types materials preferred by the customer, and providing service offers for device or new device offer with discount.

In some examples, dental device 110 may be configured to transmit raw data generated by the one or more sensors. In some examples, dental device 110 may be configured to store data 142, for example, prior to transmitting data 142 via narrowband network 140. In some examples, dental device 110 may be configured to process data 142 prior to transmitting data 142 via narrowband network 140. For example, dental device 110 may be configured to perform an analysis of data 142, such as filtering of data 142, determining a condition of dental device 110, determining a health of dental device 110, determining a device management operation, or outputting a notification.

Dental device 110 is configured to transmit data 142 via narrowband network 140. The narrowband network 140 may include any suitable low-power wide-area network (LPWAN). In some examples, narrowband network 140 may include narrowband internet of things (NB-IoT) radio technology. For example, narrowband network 140 may include a bandwidth of about 200 kHz (e.g., within the common tolerances, such as per 3GPP standards). In some examples, narrowband network 140 may include long-term evolution machine type communication (LTE-MTC). Generally, narrowband network 140 may be utilized in any suitable manner. In some examples, narrowband network 140 may include a standalone mode operation, which occurs in frequency ranges where cellular services are not present or decommissioned. In some examples, narrowband network 140 may include a guard-band mode where the Long-Term Evolution (LTE) base stations allocate narrowband access in their frequency guard bands. In some examples, narrowband network 140 may use frequencies where cellular services are present and the narrowband network shares LTE resources. In some examples, narrowband network 140 may enable communication in locations where cellular service may not be available, such as basement rooms of buildings.

Computing device 150 is configured to receive data 142 via narrowband network 140. Computing device 150 may be configured to provide data storage, data analysis, data analytics, notification generation, and/or message generation via SMS or email via cloud network. Computing device 150 may be a cloud computing device, that is located at a geographically separate location remote from dental device 110 and computing devices 180. Although shown for purposes of example as a single computing device, in some examples computing device 150 may include multiple communicatively coupled computing devices, such as part of a cloud computing system. In some examples, computing device 150 may include, but is not limited to a portable or mobile computing device (e.g., smartphone, wearable computing device, tablet), a laptop computer, a desktop computer, smart television platforms, one or more servers, a manufacturer database, a service center database, a service technician database, and/or a customer notification system.

Computing device 150 may include processing circuitry and a memory. The processing circuitry may be configured to receive data 142. The memory may include instructions that, when executed by the processing circuitry, cause the processing circuitry to determine, based on data 142, the health of dental device 110. For example, the health of dental device 110 may be based on a predetermined threshold associated with data 142. The instructions also may cause the processing circuitry to determine, based on the health, a device management operation. The device management operation may be based on analysis of data 142 and include any suitable operation configured to maintain an unimpaired health of dental device 110 or return an impaired health of dental device 110 to an unimpaired state. This may include, for example, localization of the dental device, initializing remote maintenance of dental device 110 or a component thereof, initializing repair or replacement of dental device 110 or a component thereof, improve the condition of dental device 110 or a component thereof, delivering a recommendation to a user, scheduling service, ordering replacement parts or replacement devices, providing information about use and/or care, recommending the dental device be taken out of service, or the like. By automatically generating the device management operation, customers, service centers, and/or service technicians may not need to manually identify the condition of dental device 110, the user may not need to send dental device 110 to a service center, retailer, or manufacturer, or both.

The instructions also may cause the processing circuitry to output a notification indicative of the device management operation. For example, computing device 150 may be configured to output a notification 170, for example, an alert, a text message via short message service (SMS), an automated phone call, an email, and/or notification through software or to an error light (e.g. blinking LED) or an integrated display of the dental device 110. For example, a blinking light on dental device 110 may indicate that a message has been sent via email or SMS providing information. The output notification 170 is transmitted to one or more computing devices 180, and may be output for display to a user by computing devices 180. Computing devices 180 may include one or more of a portable or mobile computing device (e.g., smartphone, wearable computing device, tablet), a laptop computer, a desktop computer, smart television platforms, a dashboard, or other computing devices. The notification may be output automatically, or generated automatically for review by, for example, a service technician, prior to outputting the notification. In some examples, the notification may be configured to offer servicing of the dental device, order replacement parts or replacement devices, recommend the user take the dental device out of service, provide analytics and/or historical data to a manufacturer or service center usable to improve a performance or a useable lifetime of dental device 110, providing a forecasted life expectancy of dental device 110, or the like. For example, historical device data may be stored and analyzed in a cloud computing system (e.g., including computing device 150), and computing device 150 may predict when dental device 110 may fail based on analysis of the historical data. In this way, dental device system may proactively inform a user of issues with dental device 110, take action to correct issues earlier and with more specificity compared to other dental device systems, or both.

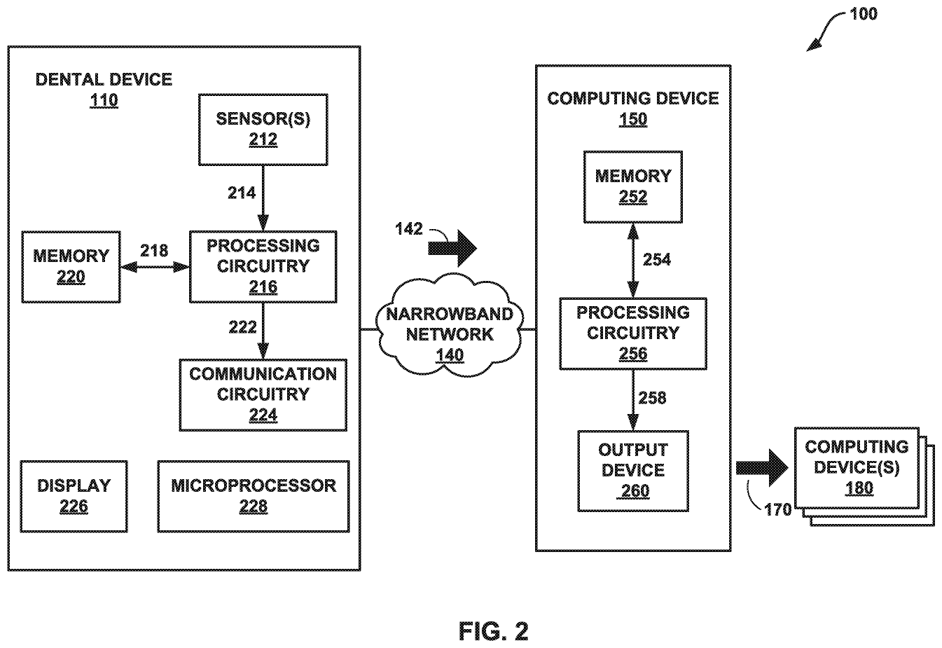

FIG. 2 is a block diagram illustrating in further detail the example dental device system 100 of FIG. 1. As illustrated in FIG. 2, dental device 110 may include one or more sensors 212 (“sensor 212”), processing circuitry 216, memory 220, communication circuitry 224, display 226, and/or a microprocessor 228. Sensor 212 is configured to generate a signal 214 indicative of a condition of the dental device 110, as discussed above. Sensor 212 may include any suitable sensor.

In some examples, sensor 212 may include a temperature sensor. The temperature sensor may be configured to measure the temperature of dental device 110 or a component thereof. For example, sensors 212 may measure a temperature of a battery, motor, circuitry, and/or other internal or external components of dental device 110. In some examples, a temperature of internal or external components of dental device 110 may be indicative of a condition of dental device 110. For example, the temperature may indicate whether dental device 110 is overheating, malfunctioning, or performing as desired.

In some examples, sensor 212 may include an accelerometer, such as a micro-electromechanical system (MEMS) accelerometer. The accelerometer may be configured to detect mechanical impact to dental device 110. Example mechanical impacts may include when dental device 110 is dropped from a selected height to the floor or otherwise contacts another object with sufficient force that internal and/or external components of dental device 110 may be damaged. In this way, sensor 212 may detect mechanical impact to dental device 110 to generate a signal indicative of a condition of dental device 110.

In some examples, sensor 212 may include a pressure transducer. The pressure transducer may include any suitable type of pressure transducer, such as piezoresistive, capacitive, potentiometric, resonant wire, magnetic, optical, or strain gauge. The pressure transducer may be configured to measure the pressure on or within one or more components of dental device 110. In some examples, the pressure on or within one or more components may be indicative of whether the one or more components are functioning properly. For example, when dental device 110 includes a post-processing station dental device, sensor 212 may be configured to detect and measure a pressure within a gas cartridge or solvent cartridge of the post-processing station dental device.

In some examples, sensor 212 may include a voltage sensor, power meter, counter, and/or error reader. The voltage sensor may be configured to measure a charge of a battery of dental device 110. The power meter may be configured to indicate, for example, device performance or operating conditions by detecting various characteristics of energy consumption. A certain pattern of energy consumption may indicate, for example, that the user may have exceeded the duty cycle of the device. Exceeding an upper limit for energy consumption may indicate a defect of a drive system, bearings, a gear drive, a motor, or the like. Exceeding a lower limit may indicate a failure of a light source, heat source, or the like. The counter may be configured to measure charging cycles or a duration the dental device system is operating. The error reader may be configured to receive device error signals or other information, for example, from microprocessor 228 or another component of dental device 110. In some examples, sensor 212 may include one or more electronic sensor configured to detect, for example, voltage, current, or impedance associated with one or more components of dental device 110. In some examples, the voltage, current, or impedance associated with one or more components may be indicative of whether the one or more components are functioning properly. In this way, sensor 212 may be configured to detect electrical signals associated with a component of dental device 110 to generate a signal indicative of a condition of dental device 110.

In some examples, sensor 212 may include one or more light meters. The light meter may be configured to detect the characteristics of a light source within the dental device 110. For example, the characteristics of a light source, e.g., a curing light, may include an intensity, a polarization, or a spectrum of light. In some examples, the characteristics of the light source may be indicative of whether the light source or a component of the light source, such as a light guide, is functioning properly. For example, a light guide may break due to mechanical impact. In this way, sensor 212 may be configured to detect characteristics of a light source associated with a component of dental device 110 to generate a signal indicative of a condition of dental device 110.

In some examples, sensor 212 may include an acoustic sensor. The acoustic sensor may be configured to indicate a frequency and/or an amplitude of noise generated by one or more components of dental device 110. In some examples, the frequency and/or an amplitude of noise generated by one or more components may be indicative of whether the one or more components is functioning properly. For example, a change in a frequency and/or an amplitude of noise generated by a gear-drive or other mechanical component of dental device 110 may indicate wear of the gear-drive or other mechanical component. In this way, sensor 212 may be configured to detect noise associated with a component of dental device 110 to generate signal indicative of a condition of dental device 110.

In some examples, the sensor 212 includes a radio-frequency identification (RFID) sensor. In some examples, an RFID reader may be configured to track a manufacturing process or lifecycle of a component of dental device 110 or dental device 110 itself. In some examples, the RFID sensor may be configured to read a material or cartridge label or detect material consumption. For example, a material cartridge for using in a mixing machine may include an RFID tag readable by an RFID sensor of dental device 110. In this way, sensor 212 may be configured to detect an RFID tag to determine quantities of materials or variations in materials being used with dental device 110 to generate signal 214 indicative of a condition of dental device 110.

The dental device 110 may include sensors that are not mentioned in the examples included, but still within the scope of this disclosure. This includes multiple sensors used independently, or in combination, to generate a signal 214 indicative of condition of dental device 110.

In some examples the dental device 110 may include processing circuitry 216. Processing circuitry 216 may be configured to receive signal 214 from sensor 212. In some examples, processing circuitry 214 may transmit data 218 to memory 220. Memory 220 may include one or more storage devices configured to store information for processing during operation of computing device 150. In some examples, memory 220 may include a volatile memory (e.g., such as random access memories (RAM), dynamic random access memories (DRAM), static random access memories (SRAM), and other forms of volatile memories known in the art) and/or a non-volatile memory (e.g., magnetic hard discs, optical discs, floppy discs, flash memories, or forms of electrically programmable memories (EPROM) or electrically erasable and programmable (EEPROM) memories). Memory 220 may be an allocation of memory contained within the processing circuitry 216. Additionally, or alternatively, memory 20 may be a separate component within the dental device 110, used by the processing circuitry 216 either solely or with other components of dental device 110.

Data 218 may include instructions that, when executed by processing circuitry 216, cause processing circuitry 216 to generate and output data 222 to the communication circuitry 224. Data 222 may be based on signal 214 indicative of a condition of dental device 110. In some examples, data 222 may be indicative of a health of the dental device 110, a device management operation, and/or a notification based on a condition or a health of dental device 110, or a device management operation. In some examples, processing circuitry 216 may include or be communicatively coupled to a microcontroller, microprocessor, and/or other electrical components used to determine and output data 222 indicative of the health of dental device 110. In some examples, processing circuitry 216 may be configured to perform an analysis of data 214, such as filtering of data 214, determining a condition of dental device 110, determining a health of dental device 110, determining a device management operation, or outputting a notification via display 226. The processing circuitry 216 outputs data 222 indicative of the health of the dental device 110 to the communication circuitry 224.

In some examples, the dental device 110 may not include processing circuitry 216 and/or memory 220. For example, sensor 212 may transmit a signal or data indicative of the condition or the health of dental device 110 to the communication circuitry 224. For example, sensor 212 may include circuitry configured to condition or process an analog signal or a digital signal generated by sensor 212 into data 222 indicative of the health of the dental device 110.

Communication circuitry 224 transmits data 142 via a narrowband network 140 to processing circuitry 256 of computing device 150. In some examples, communication circuitry 224 may be configured to condition signal 214 and/or data 222 prior to transmission as data 142. For example, communication circuitry 224 may condition signal 214 and/or data 222 for transmission via a selected frequency. As another example, communication circuitry 224 may filter at least a portion of signal 214 and/or data 222 to reduce an amount of data for transmission. In this way, communication circuitry may increase the speed or efficiency of data transmission from dental device 110 to computing device 150.

Computing device 150 receives the data 142 via the narrowband network 140 from the communication circuitry 224. As illustrated in FIG. 2, computing device 150 includes memory 252, processing circuitry 256, and output device 260. In other examples, computing device 150 may include fewer components or additional components. In some examples, processing circuitry 256 includes a microcontroller, microprocessor, and/or other electrical components used to determine a device management operation 258. The memory 252 in some examples may be an allocation of memory within the computing device 150, contained within the processing circuitry 256, and/or a separate component within the computing device 150, used by the processing circuitry 256 either solely or with other components of computing device 150. Output device 260 may include any suitable device to transmit the determined device management operation 258 to an external device, such as computing devices 180. Output device 260 may be configured to provide output to a user using audio or video media. For example, output device 260 may include a display, a sound card, a video graphics adapter card, or any other type of device for converting a signal into an appropriate form understandable to humans or machines. In some examples, output device 260 may be communicatively coupled to a wireless network connection, a narrowband network connection, a cable connection, and/or other devices used to output data.

Processing circuitry 256 of computing device 150 receives data 142 from communication circuitry 224 of dental device 110 via the narrowband network 140. After receiving data 142, processing circuitry 256 may process data 142. For example, processing circuitry 256 may reconstruct data 142. Processing circuitry 256 may communicate data 254 to and from the memory 252 to output a device management operation 258 to the output device 260. For example, memory 252 may include instructions that, when executed by the processing circuitry 256, cause processing circuitry 256 to output a device management operation 258. The instructions within the memory 252 may include, but are not limited to, a lookup table, a functional model equation, a state model, logical model, and/or other methods for generating one or more outputs (e.g., data 258) from a set of one or more inputs (e.g., data 142 and/or data 254). The processing circuitry 256, carrying out the instructions from the memory 252, may output device management operation 258 to output device 260. Output device 260 may output a notification 170 indicative of device management operation 258 to the one or more computing devices 180.

In some examples, notification 170 indicative of device management operation 258 may include, but is not limited to, an alert, a text message via short message service (SMS), an automated phone call, an email, and/or notification through software or to a display 226 of the dental device 110. Example computing devices 180 include, but are not limited to, a personal computer, a telephone, a smartphone, a manufacturer database, a service technician database, and/or a customer notification system. The example dental device system 100 in FIG. 2 is not limited to the examples mentioned, as other example dental device systems are within the scope of this disclosure.

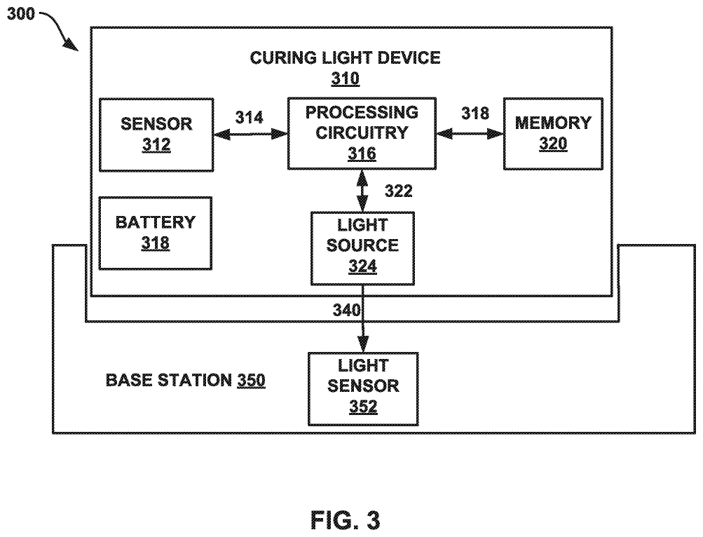

FIG. 3 illustrates an example dental device system 300 including a curing light dental device 310 (“curing light 310”) that transmits optical data 340 indicative of a condition of curing light 310 via a light source 324 to a base station 350.

Base station 350 is configured to receive optical data 340 with a light sensor 352. In some examples, base station 350 is configured to receive the curing light 310. For example, curing light 310 may be configured to dock with base station 350 to charge a battery 318 of curing light 310. In some examples, curing light 310 may be directly coupled to the base station 350 via a wired connection or tether. Curing light 310 and base station 350 may be oriented at any suitable angle. For example, the base station 350 may be orientated so that its longest dimension is parallel to the surface it rests on, e.g., so the curing light 310 is in the horizontal position, or the base station 350 may be orientated so that its longest dimension is perpendicular to the surface it rests on, e.g., the curing light 310 is received in the base station vertically.

Curing light 310 may be the same as or substantially similar to dental device 110 described above in reference to FIGS. 1 and 2, except for the differences described herein. For example, curing light 310 includes one or more sensors 312 (“sensor 312”), processing circuitry 316, and memory 320. Sensor 312 may be configured to generate a signal 314 indicative of a condition of curing light 310. Sensor 312 may include any one or more of sensors 212 described above in reference to FIG. 2. For example, curing light 310 may include a temperature sensor used to determine whether the battery 318 is increasing in temperature during charging, a counter configured to measure the number of charging cycles, an accelerometer to detect whether curing light 310 experiences a mechanical impact, and/or a sensor configured to read device error signals or other information from a microcontroller or other component within curing light 310.

Processing circuitry 316 is configured to receive signal 314. As discussed above with respect to dental device 210, processing circuitry 316 may be configured to perform an analysis on signal 314. In some examples, processing circuitry 316 may transmit data 318 with memory 320. Data 318 may include, for example, data indicative of signal 314 or instructions that, when executed by processing circuitry 316, cause processing circuitry 316 to generate and output data 322 to the light source 324. Data 222 may be based on signal 314 indicative of a condition of curing light 310.

Light source 324 of curing light device 310 may be configured to cure dental materials. Light source 324 may be configured to emit a selected wavelength of radiation for a selected duration. Light source 324 may include any suitable type of light source. In some examples, light source may include at least one of a quartz-tungsten-halogen light, a plasma-arc light, an argon laser, or a light-emitting diode (LED).

In some examples, sensor 312 is configured to generate signal 314 indicative of a condition of curing light 310. Signal 314 may cause light source 324 to emit optical data 340 indicative of signal 314. Based on signal 314, light source 324 may transmit data indicative of a condition or a health of curing light 310 to the light sensor 352 of base station 350 as optical data 340. Optical data 340 may include, for example, data conveyed via one or more of a pattern of light flashes (e.g., a pattern of relatively shorter flashes and relatively longer light flashes), a duration of one or more light flashes, an intensity of light flashes, a spectrum of light flashes, or the like. In some examples, processing circuitry 316 is configured to select one or more of a particular pattern of light flashes, an intensity of light flashes, a spectrum of light flashes, a combination thereof, or other characteristics of light, to convey optical data 340. In some examples, a characteristic optical signal (e.g., having a particular intensity, pattern, and/or spectrum of light) may be indicative of a selected condition of curing light 310. For example, processing circuitry 316 may select (e.g., based on mapping data, or the like) a different characteristics of an optical signal to convey different optical data. In some examples, memory 320 may store a data structure that includes mapping data that maps sensed signals 314 to characteristic optical signals to be emitted as optical data 340.

As one example, during operation, at a first time sensor 312 may generate a first signal (e.g., signal 314) indicative of an unimpaired health of curing light 310 (or a condition indicative of an unimpaired health of curing light 310). In response to generating the first signal, curing light 310, e.g., processing circuitry 316 and memory 320, may select a first characteristic optical signal. Processing circuitry 316 may cause light source 324 to transmit the first characteristic optical signal to light sensor 352 of base station 350 as optical data 340. At a second time (e.g., a duration after the first time), sensor 312 may generate a second signal (e.g., signal 314) indicative of an impaired health of curing light 310 (or a condition indicative of an impaired health of curing light 310). In response to generating the second signal, curing light 310, e.g., processing circuitry 316, may select a second characteristic optical signal. Processing circuitry 316 may cause light source 324 to transmit the second characteristic optical signal to light sensor 352 of base station 350 as optical data 340.

In some examples, base station 350 may include processing circuitry and/or memory, as discussed above in reference to FIG. 2, to generate data indicative of a health of curing light 310 based on optical data 340. In some examples, curing light 310 may contain processing circuitry and/or memory to generate data 314 indicative of a health of curing light 310 based on signal 312 and transmitting data 314 to the light source 324, as described in FIG. 2.

Base station 350 receives the data 340 via the light sensor 352. In some examples, the light sensor 352 includes individually, or in combination, a photoresistor, photodiode, photovoltaic, phototransistor, and/or other light sensing components. The light sensor 352, in some examples, may be configured to measure the intensity of the light source 324 to determine the health of the light source 324. In some examples, base station 350 may include additional components (not shown) configured to transmit data (e.g., data 142) indicative of optical data 340 via a narrowband network to a computing device, e.g., computing device 150, as discussed above. For example, as described in the example dental device system 100 in FIG. 2, the curing light device system 300 may include processing circuitry, memory, and/or communication circuitry configured to transmit data indicative of the condition of the curing light device 310 via the narrowband network to a computing device to generate data indicative of the health of the curing light device system 300.

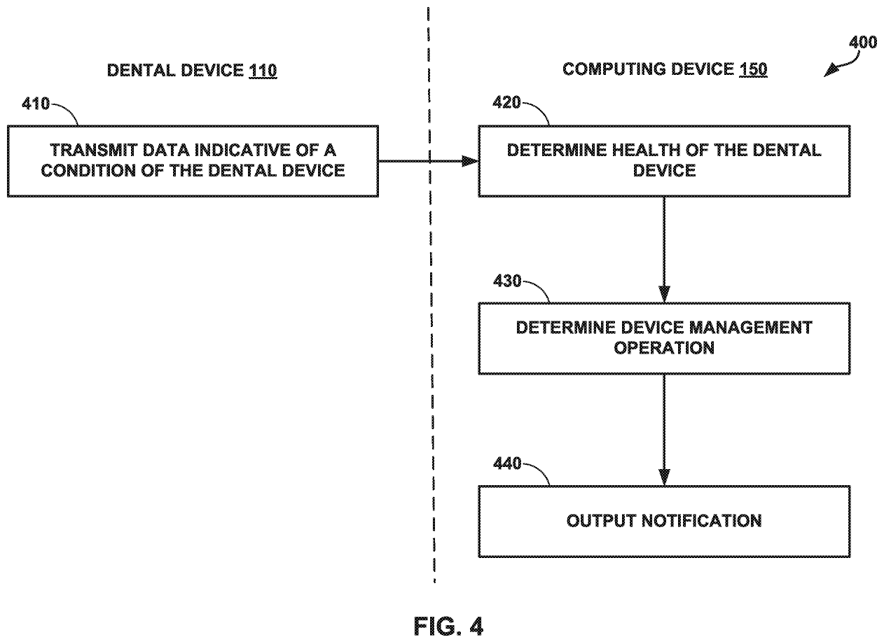

FIG. 4 is a flow chart illustrating an example technique 400 for outputting a notification 440 indicative of a device management operation 430 for a dental device 110. Although the technique illustrated in FIG. 4 is described in reference to dental device system 100 illustrated in FIGS. 1 and 2, the technique may be used with other systems, such as dental device system 300. Additionally, dental device systems 100 and 300 may be used with other techniques for outputting a notification indicative of a device management operation for a dental device.

The technique illustrated in FIG. 4 includes transmitting, by dental device 100, e.g., communication circuitry 224, data indicative of a condition 410 of the dental device to computing device 150 (410). In some examples, the data may include a digital or analog signal, e.g., signal 214 generated by sensor 212.

The technique illustrated in FIG. 4 also includes determining, by computing device 150, e.g., processing circuitry 256, a health of the dental device (420). In some examples, determining the health of dental device 110 may include determining, by processing circuitry 216, the health of the dental device prior to transmitting data to computing device 150. The health of dental device 110 may include, for example, an operating status of dental device 110, a performance metric associated with dental device 110, a failure state of a component of dental device 110, or an indication of misuse of dental device 110.

The technique illustrated in FIG. 4 also includes, in response to determining the health of dental device 110, determining, by computing device 150, e.g., processing circuitry 256, a device management operation (430). In some examples, determining the device management operation may include generating a service plan designed to correct an issue associated with the health of dental device 110. This way a service technician is more able to quickly service dental device 110 and/or ensure proper maintenance is performed on dental device 110. In some examples, determining the device management operation may include automatically generating a delivery of a replacement of a component of dental device 110 or product to replace dental device 110. By automatically generating a replacement delivery, the technique may reduce downtime or lost productivity due to malfunction or inoperability of dental device 110. In some examples, determining the device management operation may include generating or modifying a guaranty package or warranty package associated with the dental device or a component of the dental device. In some examples, determining the device management operation 430 may include determining a failure rate of dental device 110 or a component of dental device 110. In some examples, determining the device management operation may include determining a usage rate of dental device 110 or a usage of materials with dental device 110. In these ways, the technique allows manufacturers, retailers, and/or service technicians of dental device 110 to ensure better customer satisfaction and allow for the design and production of more robust dental device 110.

The technique illustrated in FIG. 4 also includes, based on the determined device management operation, notifying, by computing device 150, e.g., output device 260, a notification to computing device 180. Outputting the notification may include outputting any suitable notification that may be receivable by a human, such as an operator of the dental device 110, or a machine, such as a computing system of a dental office. For example, outputting the notification may include outputting one or more of an alert, a text message via short message service (SMS), an automated phone call, an email, notification through software or to an integrated display of dental device 110, or the like. As discussed above, computing device 180 may include a smartphone, a personal computer, and/or other computing device, e.g., having a display or other output device that outputs visual or audible information for a user to understand what device management operation should be performed on dental device 110.

Various examples have been described. These and other examples are within the scope of the following claims.

1. A dental device system comprising:
a computing device; and
a dental device comprising:
a sensor configured to generate a signal indicative of a condition of the dental device; and
communication circuitry configured to receive the signal from the sensor, the communication circuitry configured to transmit data indicative of a health of the dental device via a narrowband network to the computing device,
wherein the computing device comprises:
processing circuitry configured to receive the data indicative of the health of the dental device via the narrowband network; and
a memory configured to store instructions that, when executed by the processing circuitry, cause the processing circuitry to:
determine, based on the data, the health of the dental device;
determine, based on the health, a device management operation; and
output a notification indicative of the device management operation.

2. The dental device system of claim 1, wherein the dental device further comprises processing circuitry of the dental device, the processing circuitry of the dental device being configured to:
receive the signal from the sensor; and
process the signal to generate the data indicative of the health of the dental device system.

3. The dental device system of claim 1, wherein the sensor comprises at least one of a temperature sensor, an accelerometer, a pressure sensor, a voltage sensor, a power meter, a luminance sensor, an acoustic sensor, a radio-frequency identification sensor, or a counter configured to measure charging cycles or a duration the dental device system is operating.

4. The dental device system of claim 1, wherein the dental device comprises a curing light device having a light source and a battery.

5. The dental device system of claim 4, wherein the condition of the dental device comprises at least one of a remaining battery life of the battery, a total number of charging cycles of the battery, a battery voltage, a mechanical impact to the curing light device, a temperature around the battery or around the light source or around electronics of the dental device, a luminance of the light source, a number of on/off cycles of the light source, or a duration of operation of the curing light device.

6. The dental device system of claim 4, wherein the dental device system further comprises a base station configured to receive the curing light device, wherein the base station comprises a light sensor configured to detect light emitted from the light source.

7. The dental device system of claim 6, wherein the curing light device is configured to transmit data to the base station as a pattern of light flashes.

8. The dental device system of claim 6, wherein the light sensor is configured to detect an intensity of the light emitted from the light source.

9. The dental device system of claim 1, wherein the dental device comprises a mixing machine having at least one motor configured to drive a gear-drive or an output shaft to mix at least two materials, wherein the at least two materials come from respective material canisters or are stored and mixed in a canister with two chambers.

10. The dental device system of claim 9, wherein the condition of the dental device comprises at least one of a mechanical impact to the mixing machine, a temperature of the motor or the gear-drive or motor controller circuitry, a duration of operation of the motor or the gear-drive, the acoustics of the device, a pressure at the respective material canisters, or a type of material extruded through the mixer.

11. The dental device system of claim 1, wherein the dental device comprises a post-processing station configured to post-treat at least one of dental products or dental instruments using at least one of a cleaning fluid, heat, acceleration, abrasion, or radiation.

12. The dental device system of claim 11, wherein the condition of the dental device comprises at least one of a mechanical impact to the post-processing station, a temperature of the motor or the gear-drive or motor controller circuitry, a duration of operation of the motor or the gear-drive or light source or heat source, acoustics of the dental device, a type of cleaning fluid used in the post-processing station, a count of cleaning cycles, a duration of cleaning cycles, or a pressure or vacuum within post-processing station.

13. The dental device system of claim 1, wherein the dental device system comprises a three-dimensional printer configured to receive a material cartridge for printing via applying or providing the material from a cartridge to additively build a dental appliance.

14. The dental device system of claim 13, wherein the condition of the dental device comprises at least one of a mechanical impact to the three-dimensional printer, a type of material cartridge used in the three-dimensional printer, a leakage of the cartridge or printer, a temperature of the printing head or inside the printer, acoustics associated with movement inside the printer, a luminance of the light source of the three-dimensional printer or a duration of operation of the three-dimensional printer.

15. The dental device system of claim 1, wherein the sensor comprises a first sensor, wherein the dental device further comprises a second sensor, wherein the second sensor is configured to generate at least a second signal indicative of at least a second condition of the dental device.

16. A dental device comprising:
a sensor configured to generate a signal indicative of a condition of the dental device;
processing circuitry configured to:
receive, from the sensor, the signal; and
process the signal to generate data indicative of a health of the dental device; and
communication circuitry configured to transmit the data via a narrowband network.

17. The dental device system of claim 16, wherein the sensor comprises at least one of a temperature sensor, an accelerometer, a pressure sensor, a voltage sensor, a power meter, a luminance sensor, an acoustic sensor, a radio-frequency identification sensor, or a counter configured to measure charging cycles or a duration the dental device system is operating.

18. The dental device system of claim 16, wherein the dental device comprises a curing light device having a light source and a battery.

19. The dental device system of claim 18, wherein the condition of the dental device comprises at least one of a remaining battery life of the battery, a total number of charging cycles of the battery, a battery voltage, a mechanical impact to the curing light device, a temperature around the battery or around the light source or around electronics of the dental device, a luminance of the light source, a number of on/off cycles of the light source, or a duration of operation of the curing light device.

20-29. (canceled)

30. A computing device comprising:
processing circuitry; and
a memory comprising instructions that, when executed by the processing circuitry, cause the processing circuitry to:
receive data indicative of a condition of a dental device via a narrowband network;
determine, based on the data, a health of the dental device;
determine, based on the health, a device management operation; and
output a notification indicative of the device management operation.

31-42. (canceled)
